(19) United States
(12) Patent Application Publication
(10) Pub. No.: US 2017/0326208 A1
(43) Pub. Date: Nov. 16, 2017

(54) TREATMENT OF DISEASES ASSSOCIATED WITH FAT ACCUMULATION

(71) Applicant: The Provost, Fellows, Foundation Scholars, & the other members of the Board, of the College of the, Dublin (IE)

(72) Inventors: Padraic FALLON, Dublin (IE); Emily HAMS, Dublin (IE)

(21) Appl. No.: 15/532,827

(22) PCT Filed: Dec. 3, 2015
(86) PCT No.: PCT/EP2015/078578

§ 371 Date: Jun. 2, 2017

(30) Foreign Application Priority Data
Dec. 4, 2014 (EP) 14196247.2

(51) Int. Cl. A61K 38/46

(52) U.S. Cl. CPC A61K 38/465; C12Y 301/27001

(57) ABSTRACT

The present invention is directed to immunmodulators in the form of compositions, compounds, proteins and/or fragments with RNase activity thereof for use in the treatment of diseases associated with fat accumulation, including obesity and obesity-related disorders and metabolic disorders.

The present invention is directed to immunmodulators in the form of compositions, compounds, proteins and/or fragments thereof for use in the treatment of diseases associated with fat accumulation, including obesity and obesity-related disorders and metabolic disorders.

### BACKGROUND

Obesity has become a worldwide epidemic with recent WHO statistics suggesting that approximately 500 million adults and 43 million children under the age of 5 are considered obese. Obesity is often associated with several co-morbidities, including the development of metabolic syndrome, which carries significant risks for the development of cardiovascular disease. The obesity epidemic is a serious burden on the health system, costing the Irish government approximately [symbol] 400 million in 2012.

A hallmark of obesity is chronic low-grade inflammation that has a pivotal role in the progression to metabolic disorders, such as atherosclerosis and type 2-diabetes. It is known that the inflammatory cell composition of adipose tissue can profoundly influence the regulation of weight and the maintenance of metabolic homeostasis. While ‘lean’ adipose tissue is populated with anti-inflammatory cells such as eosinophils, alternatively activated macrophages (AAM) and regulatory T cells (Treg), increase in dietary fats and sugars can induce an inflammatory response within the adipose tissue resulting in recruitment of cytotoxic CD8+ T cells and classically activated macrophages (CAM).

While studies into homeostatic glucose regulation have outlined the importance of AAM in promoting insulin sensitivity, eosinophils have been shown to be important in sustaining AAM in the visceral adipose tissue (VAT) of mice on HFD, by the localized release of interleukin IL-4 and IL-13. More recently attention has focused on the role of innate lymphoid cell types (ILC) that are present in the VAT, in particular type 2 ILC. Interestingly one such ILC2 population was identified in fat-associated lymphoid clusters in both mice and humans.

A number of drugs are being developed as immunotherapies for the management of obesity (1). Additionally, the weight loss caused by the glucagon-like peptide 1 agonist (Liraglutide), used for the treatment of type 2 diabetes mellitus, has been shown to be associated with immune modulation (2).

The ability of a type 2 response to have such profound effects on insulin sensitivity and glucose tolerance has led to the search for an appropriate molecule to effectively and robustly induce a type 2 response in the adipose tissue as an anti-obesity therapy. For example, recent studies in mice have identified roles for ILC2, induced in response to helminth infection, in the localization of eosinophils within visceral adipose tissue (VAT) and the local expansion of AAM.

Furthermore, live helminth infection of obese mice has been found to induce weight loss by the propensity to induce type 2 responses [3]. It has also been found that eggs from the helminth parasite Schistosoma mansoni are potent natural inducers of a type 2 response [4]. Recent studies have identified a glycosylated T2 RNase, named omega-1 (ω1), as the primary component in S. mansoni eggs responsible for initiating and driving a type 2 response [5, 6]. Omega 1 is 31 kDA glycoprotein secreted from eggs containing a 36 bp 5′-untranslated region, a 675 bp coding region (coding for 225 amino acids), a 48 bp 3′-untranslated region and a poly A tail (GENBANK: DQ013208) [7, 8].

Recent studies on ω1 (omega-1) have focused on the in-vitro ability of ω1 to drive dendritic cells (DC) to polarize naïve CD4 cells to a IL-4 and IL-5 producing Th2 cell phenotype, via a mechanism requiring both its glycosylation and RNase activity [9]. While glycosylation is required for internalization of ω1 via binding to the mannose receptor (CD206), the RNase interferes with protein synthesis by global cleavage of rRNA and mRNA once translocated to the cytosol, enabling ω1 to condition DCs for priming of Th2 cell expansion [9]. However, these studies have only focused on characterizing ω1 and the identification of how, at the cellular level, ω1 can initiate a Th2 cell response, based on release of IL-4 and IL-5 under in-vitro conditions and increase in IL-4+CD4 cells in vivo. No other information is known about ω1.

The present invention is directed to providing a new therapy for obesity and diseases associated with fat accumulation.

### STATEMENTS OF THE INVENTION

According to a general aspect of the invention, there is provided a compound, protein or fragment thereof that induces cytokine IL-33 release to initiate a type 2 inflammatory response for use in the treatment of diseases associated with fat accumulation. It will be understood that the compound or protein or fragment thereof ideally has ribonuclease activity. Ideally, the compound or protein of the invention is an immunomodulatory or adjuvant compound or protein. Alternatively, the compound or protein of the invention may be administered as a combination therapy, either simultaneously or sequentially, with another active ingredient, such as conventional or other obesity therapy.

According to a first aspect of the invention, there is provided a ribonuclease protein or ribonuclease-like protein, preferably a ribonuclease protein of the T2 family, or fragment thereof that induces IL-33 release to initiate a type 2 inflammatory response, preferably in adipose cells and/or tissues, for use in the treatment of diseases associated with fat accumulation. Ideally, the protein is Omega-1 protein, advantageously derived from Schistosoma mansoni eggs, or a fragment thereof.

According to a second aspect of the invention, there is provided a pharmaceutical composition comprising a compound, protein or fragment thereof that induces IL-33 release to initiate a type 2 inflammatory response, preferably in adipose cells and/or tissues, for use in the treatment of diseases associated with fat accumulation. It will be understood that the compound or protein or fragment thereof ideally has ribonuclease activity. Ideally, the compound or protein of this aspect of the invention is an immunomodulatory compound or protein. Optionally, the protein is a ribonuclease protein, preferably a ribonuclease protein of the T2 family, more preferably an Omega-1 protein, advantageously derived from Schistosoma mansoni eggs, or a fragment thereof.

According to a third aspect of the invention, there is provided a method for the treatment of diseases, preferably diseases associated with fat accumulation, comprising the administration of a compound, protein or fragment thereof that induces IL-33 release to initiate a type 2 response, preferably in adipose cells and/or tissues, to a subject in need thereof. It will be understood that the compound or protein or fragment thereof ideally has ribonuclease activity. Ideally, the compound or protein of this aspect of the invention is an immunomodulatory compound or protein. Optionally, the protein is a ribonuclease protein, preferably a ribonuclease protein of the T2 family, more preferably an Omega-1 protein, advantageously derived from Schistosoma mansoni eggs, or a fragment thereof.

According to a fourth aspect of the invention, there is provided the use of a compound, protein or fragment thereof that induces IL-33 release to initiate a type 2 inflammatory response, preferably in adipose cells and/or tissues, for use in the manufacture of a medicament for the treatment of diseases, associated with fat accumulation. It will be understood that the compound or protein or fragment thereof ideally has ribonuclease activity. Ideally, the compound or protein of this aspect of the invention is an immunomodulatory compound or protein. Optionally, the protein is a ribonuclease protein, preferably a ribonuclease protein of the T2 family, more preferably an Omega-1 protein, advantageously derived from Schistosoma mansoni eggs, or a fragment thereof.

### DETAILED DESCRIPTION

In this specification, it will be understood that the term “comprising” encompasses and can be replaced by the terms “comprise, comprises, comprised and comprising”, “consist, consists, consisted and consisting”, “consist essentially of, consists essentially of, consisted essentially of and consisting essentially of” and “include, includes, included and including” or any variation thereof. These terms are considered to be totally interchangeable and they should all be afforded the widest possible interpretation.

In this specification, reference to any naturally occurring protein will be understood to encompass an isolated protein and/or recombinant protein.

In this specification, it will be understood that proteins or fragments thereof with sufficiently high homology to ribonuclease proteins, such as omega-1, may also be used. High homology as defined herein occurs when at least 50%, preferably 60%, preferably 70%, preferably 80%, more preferably 90%, even more preferably 95%, still more preferably 95% to 99%, still more preferably 99% of the nucleotide or amino acid residues match over the entire length of the nucleotide or amino acid sequence. It will be understood that these comments about high homology may also relate to the 3D structure of the protein to produce a protein having the same functionality and activity.

In this specification, it will be understood that the invention embraces the claimed ribonuclease proteins or fragments thereof with defined amino acid or nucleotide residues or variants thereof including “ribonuclease-like” proteins. The term “ribonuclease-like” proteins is intended to cover proteins which have related amino acid sequences, similar modular design and/or common/similar binding domain organization to such ribonucelase proteins. For example, the ribonuclease-like proteins may have at least 50%, preferably 60%, preferably 75%, more preferably 85%, even more preferably 95%, still more preferably 99% or more amino acid sequence identity or homology with the ribonuclease proteins.

It will also be understood that any of the percentage identities or homologies referred to in the specification are determined using available conventional methods over the entire/whole length of the sequence.

In this specification, it will be understood that diseases associated with fat accumulation include obesity, obesity related disorders, liver related fat accumulation and metabolic disorders.

Obesity related disorders include but are not limited to heart disease, stroke, high blood pressure/hypertension, glucose disorders including but are not limited to diabetes (type 1 and type 2 diabetes mellitus), cancer, gallbladder disease and gallstones, osteoarthritis, gout, breathing problems, such as sleep apnea and asthma.

Metabolic disorders associated with fat accumulation include obesity related disorders and encompass diseases such as type 1 and type 2 diabetes mellitus, high blood pressure/hypertension, nonalcoholic fatty liver disease, atherosclerosis, cancers, breathing problems including but not limited to sleep apnea and cardiovascular diseases

Liver related fat accumulation disorders includes fatty liver disease.

According to a general aspect of the invention, there is provided a compound, protein or fragment thereof that induces IL-33 release to initiate a type 2 inflammatory response in adipose tissue for use in the treatment of diseases associated with fat accumulation. In this manner, the compound, protein or fragment thereof of the invention will be understood to promote accumulation of type 2 immune cells tissues, such as adipose cells and/or tissues, wherein the type 2 immune cells are ideally selected from eosinophils, innate lymphoid cells (ILC2), CD3+ T cells, basophils, mast cells, macrophages and/or alternatively activated macrophages (AAM). It will be understood that the compound or protein or fragment thereof ideally has ribonuclease activity. In this manner, the compound, protein or fragment thereof may be an isolated protein or fragment thereof or a synthetic protein or fragment designed to provide ribonuclease activity. Ideally, the compound or protein of the invention is an immunomodulatory compound or protein or adjuvant compound or protein.

According to a preferred embodiment of the invention, there is provided a ribonuclease protein or ribonuclease-like protein that induces IL-33 release to initiate a type 2 response, preferably in adipose cells and/or tissues, for use in the treatment of diseases associated with fat accumulation.

Ideally, the ribonuclease protein is an isolated protein derived from the T2 family. RNase T2 proteins are characterized by the presence of RNase active sites or catalytic domains that are conserved in every T2 protein. T2 RNases are endonucleases that cleave RNA via a 2′3′ cyclic intermediate and typically contain two conserved amino acid sequences (CAS-1 and CAS-2) that incorporate the residues critical for RNase activity.

It will be understood that other ribonuclease proteins (other than T2 family derived proteins) may be contemplated. The ribonucelase protein may be prokaryotic or eukaryotic. For example, the ribonucelase protein may be a bacterial, fungal or plant RNase protein or RNase T2 protein. The ribonuclease proteins may be specific for single-stranded RNAs or for double stranded RNAs. The ribonuclease protein may be selected from one or more of RNase A, H, I, III, L, P, PhyM, T1, T2, U2, V1, and/or V. Alternatively, the ribonuclease protein may selected from one or more of RNase PH, II, R, D and/or T. These proteins may be natural, isolated proteins or synthetic proteins. Proteins with at least 50%, 60%, 70%, 80%, 90%, 95%, 96%, 97%, 98% or 99% sequence homology or identity to ribonuclease proteins or RNase T2 proteins may also be contemplated.

According to a further embodiment, ribonuclease-like proteins or fragments thereof may be contemplated. Ribonuclease-like proteins are essentially proteins, other that ribonuclease proteins, which have ribonuclease activity. These proteins may be natural, isolated proteins or synthetic proteins. In this specification, ribonuclease-like proteins may be derived or isolated from from native or wild-type ribonuclease proteins. Enzymatic activity in terms of RNase activity is essential. Optionally, ribonuclease-like proteins may be synthetic proteins designed to mimic the structure or part thereof of such native or wild-type ribonuclease proteins. Again, enzymatic activity in terms of RNase activity is essential. Optionally, a ribonuclease-like protein fragment may be used.

Ideally, the ribonuclease, ribonuclease-like protein or RNase T2 protein or fragment thereof comprises one or more RNase catalytic domains.

Optionally, the ribonuclease, ribonuclease-like protein or RNase T2 protein or fragment thereof is enzymatically active with a modified N-glycolysation site or sites.

Optionally, the ribonuclease or RNase T2 protein or fragment thereof may be altered at the amino acid level by way of one or more nucleotide or amino acid deletions, insertions and/or substitutions. In this way, the N-glycosylation sites may be modified but still be enzymatically active.

Optionally, the ribonuclease or RNase T2 protein or fragment thereof may be modified to a glycoprotein, for example it may be adapted to further comprise a glycan or other backbone.

Ideally, according to all aspects of the invention, an isolated and/or recombinant protein or fragment thereof is utilized.

Preferably, according to all aspects of the invention, a purified protein is used. In this manner the native, synthesized or recombinant protein or fragment thereof may be purified using conventional techniques to remove any undesirable endotoxins or other non-protein components. For example, the native or recombinant protein or fragment thereof may be washed using a detergent or subject to chromatography. In this manner, the protein according to all aspects of the invention is endotoxin-free.

The present invention is based on the unexpected findings that a recombinant T2 RNase immunomodulator from Schistosoma mansoni eggs, omega-1, reverses obesity and restores glucose homeostasis in a mouse model of diet-induced obesity. We have unexpectedly shown that this occurs via IL-33 release and activation. This is independent to IL-4/IL-5 release and activation. Our findings that omega-1 induces IL-33 release to initiate a type 2 response in adipose tissue were unexpected because omega-1 was only previously associated with IL-4 and IL-5 activity.

Accordingly, a most preferred embodiment of the invention the protein is an Omega-1 protein or a fragment thereof. It will be understood that Omega-1 is derived from Schistosoma mansoni eggs.

According to this preferred embodiment there is provided and omega-1 protein, ideally a recombinant omega-1 protein or a fragment thereof that induces IL-33 release to initiate a type 2 response, for use in the treatment of diseases associated with fat accumulation.

These diseases that are associated with fat accumulation include
obesity related disorders are selected from heart disease, stroke, high blood pressure/hypertension, glucose disorders including diabetes (type 1 and type 2 diabetes mellitus), cancer, gallbladder disease and gallstones, osteoarthritis, gout, breathing problems, such as sleep apnea and asthma;
metabolic disorders associated with fat accumulation include type 1 and type 2 diabetes mellitus, high blood pressure/hypertension, nonalcoholic fatty liver disease, atherosclerosis, cancers, breathing problems including sleep apnea and cardiovascular diseases; and
the liver related fat accumulation disorders is fatty liver disease.

It is known that the amino acid sequence for omega-1 contains an initial signal peptide of 23 amino acids with a high probability of cleavage to yield a mature polypeptide consisting of 224 amino acids (as shown in FIG. 2/SEQ ID Nos. 1 and 2). Native omega-1 contains 2 potential N-glycosylation sites at residues 71 and 176. Studies have also identified omega-1 as a T2 RNase, with 31% homology to extracellular RNase LE, a well-characterized plant enzyme. It also comprises two conserved amino acid sequences (CAS-1 and CAS-2) that incorporate the residues critical for RNase activity. CAS1 corresponds to amino acid residues FTIHGLWPT (SEQ ID No. 3) and CAS2 (SEQ ID No. 4) corresponds to amino acid residues PSFWKHEFEKHGLCAV. According to one embodiment of the invention, the full length Omega-1 protein comprising amino acid residues 1 to 224 may be used. Based on our studies, we found that sites responsible for glycosylation, N71 and N76 were required for activity (see FIG. 2).

Alternatively, an Omega-1 protein fragment comprising at least part of amino acid residues 1 to 224 may be used.

In this manner, the Omega-1 protein or fragment thereof may be altered at the amino acid level by way of one or more nucleotide or amino acid deletions, insertions and/or substitutions.

We postulate that the N-glycosylation sites at residues 71 and 176 are essential to the invention. Although, for example, N-glycosylation sites at residues 71 and 176 may in some instances mutated. Although native wild-type omega-1 has been reported to be cytotoxic due to its RNase activity and/or its highly cationic nature. We propose that low doses of omega-1 or any RNase may be administered to a subject. Alternatively, the omega-1 or any RNase protein may be altered or changed to reduce or minimize cytotoxity. For example, omega-1 fragments may be administered. As described above, such fragments should comprise the RNase catalytic region or could potentially have altered charge. According to another embodiment, the omega-1 protein fragment has mutated N-glycolysation sites.

Wild-type Omega-1 comprises at least two RNase catalytic domain corresponding to conserved amino acid (CAS) sequence 1 and 2 (CAS1 and CAS2).

According to another embodiment, the Omega-1 fragment comprises one or more RNase catalytic domains. Accordingly, in one embodiment the Omega-1 protein fragment comprises or consists of at least a first conserved amino acid sequence (CAS1) which corresponds to amino acid residues FTIHGLWPT and/or a second conserved amino acid sequence (CAS2) which corresponds to amino acid residues PSFWKHEFEKHGLCAV. In a preferred embodiment, the Omega-1 protein fragment comprises or consists of a first conserved amino acid sequence (CAS1) FTIHGLWPT and a second conserved amino acid sequence (CAS2) PSFWKHEFEKHGLCAV.

Furthermore, the N-glycolsylation residues N71 and N176 are proposed to be essential to the invention.

As described above, Omega-1 fragments may be advantageously utilized instead of the full length protein of 224 amino acid residues. A small protein fragment can be advantageously administered to a subject. For example, such fragments may comprise approximately 10, 20, 30, 40, 50, 60, 70, 80, 90, 100, 110, 120, 130, 140, 150, 160, 170, 180, 190, 200, 210, 210 amino acids.

According to yet another embodiment of the invention, the omega-1 protein or fragment thereof is modified to comprise a glycoprotein carrier or glycoprotein backbone to maintain or enhance it functional activity. In this manner, it will also be understood that the protein or fragment may be in the form of a fusion or chimeric protein adapted for administration to a subject. For example, the fusion or chimeric protein may be adapted to target adipose cells and/or tissues.

It will also be understood that these comments relating to omega-1 fragments are equally applicable to other RNase immunomodulators or adjuvants, including other T2 RNases.

According to another general aspect of the invention, there is provided a compound, protein or fragment thereof that induces IL-33 release to initiate a type 2 response in adipose tissue for use in the treatment of obesity and obesity-related disorders and/or inducing weight loss, ideally by decreasing the number of adipose cells after administration. In this manner the compound, protein or fragment may be any immunomodulatory or adjuvant compound or protein as described above.

According to another general aspect of the invention, there is provided a compound, protein or fragment thereof that induces IL-33 release to initiate a type 2 response in adipose tissue for use in treatment of metabolic disorders, ideally by restoring glucose and insulin homeostasis after administration. In this manner the compound, protein or fragment may be any immunomodulatory or adjuvant compound or protein as described above.

According to another general aspect of the invention, there is provided a compound, protein or fragment thereof that induces IL-33 release to initiate a type 2 response in adipose tissue for use in treatment of a liver disorder, ideally by decreasing the number of adipose cells in the liver after administration. In this manner the compound, protein or fragment may be any immunomodulatory or adjuvant compound or protein as described above.

According to a second aspect of the invention there is provided a pharmaceutical composition comprising the compound, protein or fragment thereof according to any of the preceding claims. Ideally, the compound, protein or fragment thereof is administered in the form of a pharmaceutical composition further comprising a suitable conventional pharmaceutical excipient.

It will be understood that the compound, protein or fragment thereof, pharmaceutical composition according to any of the preceding claim for use as an adjuvant therapy. In this manner, the composition, compound or protein of the invention may be used alone or as an adjuvant therapy in the treatment of diseases associated with fat accumulation.

It will be understood that the compound, protein or fragment thereof of the invention may be administered as an immunomodulatory therapy alone. Alternatively, the compound, protein or fragment thereof of the invention may be administered as an adjuvant, for example at the same time (concomitant or concurrent systemic therapy) as a conventional or other obesity therapy is administered.

Many different administration routes may be contemplated including, but not limited to, oral, topical, pulmonary, rectal, subcutaneous, intradermal, intranasal, intracranial, intramuscular, intraocular, or intra-articular injection, and the like. Ideally delivery is by intravenous, intradermal, subcutaneous, intraperitoneal, intramuscular or transdermal delivery. The most typical route of administration is intravenous followed by subcutaneous, although other routes can be equally effective.

Optionally, delivery may be systemically, locally or parenterally. Oral formulations take the form of solutions, suspensions, tablets, pills, capsules, sustained-release formulations or powders. Topical application can result in transdermal or intradermal delivery. Local delivery means include direct injection to the site of interest. Systemic delivery means may include parenteral or enteral means and encompass all non-local delivery means. Systemic delivery means may include direct injection, such as intravenous injection.

In this manner, the composition, compound or protein of the invention may be administered systemically. Systemic administration may be via oral or parenteral routes.

Alternatively, the composition, compound or protein of the invention may be administered locally to adipose tissue. Local administration may be by way of injection into the adipose tissue.

According to a third aspect of the invention there is provided a method for the treatment of diseases, preferably diseases associated with fat accumulation, comprising the administration of a compound, protein or fragment thereof that induces IL-33 release to initiate a type 2 response, preferably in adipose cells and/or tissues, to a subject in need thereof. In this manner the compound, protein or fragment may be any immunomodulatory or adjuvant compound or protein as described above.

According to a fourth aspect of the invention, there is provided the use of a compound, protein or fragment thereof that induces IL-33 release to initiate a type 2 response, preferably in adipose cells and/or tissues, for use in the manufacture of a medicament for the treatment of diseases, associated with fat accumulation. In this manner the compound, protein or fragment may be any immunomodulatory or adjuvant compound or protein as described above.

The present invention will now be described with respect to the following non-limiting figures and examples.

### FIGURE LEGENDS

FIG. 1: Expression of his-tagged recombinant omega-1 from HEK-293 cells and confirmation of RNase activity. Recombinant protein was Ni-Affinity and gel filtration chromatography purified and subjected to endotoxin removal by detergent-based methods. (A) SDS-PAGE of purified proteins stained with Coomassie Blue (1) recombinant ω1; (2) RNase null ω1. (B) RNA from murine bone-marrow derived macrophages was incubated with 500 ng/ml and 100 ng/ml of recombinant ω1 and RNase null ω1 (ω1Δ^{RNase}) for 1 hour and RNA integrity analyzed on a 2% agarose gel. RNase A was used as a positive control.

FIG. 2: Nucleotide and amino acid sequence of Omega-1. N-glycosylation sites are bold; Conserved Amino acid Sequences (CAS-1 and CAS-2) are underlined.

FIG. 3: Recombinant ω1 induces weight loss and an improvement in glucose homeostasis in obese mice. (A) Weight gain, expressed as a percentage from starting weight, in WT mice on high fat diet (HFD) for 8 weeks, and treated with 25 μg ω1, or 25 μg OVA on days 0, 2 and 4. Weight was monitored for 21 days. (B) Weight of excised visceral adipose tissue (VAT) and subcutaneous adipose tissue (SAT) in OVA and ω1 treated mice at 6 days post initial injection. (C) Immunohistochemistry depicting H&E staining from excised VAT from control diet (CD) fed animals, and HFD fed animals treated with OVA and ω1. VAT was excised at day 6 post initial injection. Adipocyte area was calculated from histological slides. Blood glucose was assessed basally in fasted mice (D) and glucose tolerance assessed after injection of 2 g/kg glucose i.p. at day 6 post initial injection of r-ω1 (E). Levels of triglyceride (F) were determined in the serum of OVA and ω1 treated mice. Data are representative of n=6 (+/−SEM) from 3 independent experimental replicates (*P<0.05, ** P<0.01, ***P<0.001, ****P<0.0001).

FIG. 4: Short-term, low dose treatment with recombinant omega-1 reduces liver damage in obese mice. Mice were fed CD and HFD and treated with 25 μg endotoxin-free ω1, or 25 μg endotoxin-free OVA on days 0, 2 and 4. On day 6 AST and ALT were quantified in the serum and expressed as a ratio of AST to ALT. Data are representative of n=6 (+/−SEM) from 3 independent experimental replicates (ns—not-significant, *P<0.05).

FIG. 5: Long-term treatment with recombinant ω1 maintains weight loss and glucose homeostasis in obese mice. (A) Weight gain, expressed as a percentage from starting weight, in WT mice on high fat diet (HFD) for 8 weeks, and treated with 25 μg endotoxin-free ω1, or 25 μg endotoxin-free OVA on days 0, 2 and 4 (short-term), or on days 0, 4, 8, 12, 16 and 20 (long-term). Weight was monitored for 21 days. (B) Weight of excised VAT in OVA and ω1 treated mice at 21 days post initial injection. Blood glucose was assessed basally in fasted mice (C) and glucose tolerance assessed after injection of 2 g/kg glucose i.p. at day 21 post initial injection of ω1 (D). Data are representative of n=3 (+/−SEM) from 2 independent experimental replicates (*P<0.05, **P<0.01).

FIG. 6: Recombinant ω1 induces a type 2 immune cell repertoire in the VAT of obese mice. Cellular infiltration into the VAT of obese mice treated with 25 μg endotoxin-free ω1, or 25 μg endotoxin-free OVA on days 0, 2 and 4 was assessed by flow cytometry on day 6 post initial injection. Alternatively activated macrophages (AAM) were identified as CD11b⁺F4/80⁺CD206^{lo} and CD11b⁺F4/80^{hi}CD206^{hi} respectively. Eosinophils were identified as CD11b⁺SiglecF⁺, and ILC2 as Lineage⁻IL-7Rα⁺Sca-1⁺T1/ST2⁺KLRG1⁺. Data are representative of n=6 (+/−SEM) from 3 independent experimental replicates (*P<0.05, ** P<0.01, ***P<0.001).

FIG. 7: Recombinant ω1 induces the localized release of type 2 cytokines, including IL-33. Release of IL-4, IL-5, IL-13 (A) and IL-33 (B) were quantified in the peritoneal fluid at 1, 3, 6 and 24 hours post injection of 25 μg endotoxin-free ω1 (A; 6 hours post injection) by ELISA. Culture of mouse (C) and human (D) adipocytes in the presence of 500 ng/ml ω1 results in a peak of IL-33 production after 3 hours. Data are representative of n=4-6 (+/−SEM) from 2-3 independent experimental replicates (*P<0.05, ** P<0.01).

FIG. 8: RNase mutant ω1 does not induce significant weight loss or IL-33 induction in obese mice. (A) Weight gain, expressed as a percentage from starting weight, in WT mice on high fat diet (HFD) for 8 weeks, and treated with 25 μg endotoxin-free ω1 (WT), 25 μg endotoxin-free ω1Δ^{RNase} or 25 μg endotoxin-free OVA on days 0, 2 and 4. Weight was monitored for 6 days. (B) Weight of excised visceral adipose tissue (VAT) in OVA and WT and ω1Δ^{RNase} treated mice at 6 days post initial injection. (C) Glucose tolerance assessed after injection of 2 g/kg glucose i.p. at day 6 post initial injection of WT or ω1Δ^{RNase}, blood glucose was measured at 30, 60 and 120 minutes after injection of glucose. (Data are representative of n=5 (+/−SEM) from 2 independent experimental replicates (*P<0.05, **P<0.01, ***P<0.001).

FIG. 9: Weight loss by ω1 is mediated by RNase activity. (A) Weight gain, expressed as a percentage from starting weight, in WT mice on high fat diet (HFD) for 8 weeks, and treated with 25 μg ω1 (WT), 25 μg ω1Δ^{RNase} or 25 μg OVA on days 0, 2 and 4. Weight was monitored for 6 days. (B) Weight of E-WAT in OVA and WT and ω1Δ^{RNase} treated mice at 6 days post initial injection. (C) Glucose tolerance assessed after injection of 2 g/kg glucose i.p. at day 6 post initial injection of WT or ω1Δ^{RNase}. (D) Cellular infiltration into the E-WAT was assessed by flow cytometry 6 days after initial injection of OVA, ω1 or ω1Δ^{RNase}. AAM were identified as CD11b⁺F4/80^{hi}CD206^{hi}, eosinophils were identified as CD11b⁺SiglecF⁺ and ILC2 as Lin⁻IL-7Rα⁺Sca-1⁺T1/ST2⁺KLRG1⁺. Data are representative of n=5-8 (+/−SEM) from 2 independent experimental replicates (ns—not significant, *P<0.05, **P<0.01, ***P<0.001).

FIG. 10: Effective binding to CD206 is essential for the functional activity of ω1. Weight gain, expressed as a percentage from starting weight, in WT mice on high fat diet (HFD) for 8 weeks, and treated with 25 μg ω1 (WT), 25 μg ω1Δ^{GLY} or 25 μg OVA on days 0, 2 and 4. Data are representative of n=2-6 (+/−SEM) from 2 independent experimental replicates (ns—not significant, ** P<0.01).

FIG. 11: Hepatic steatosis assesment showing the ratio of AST to ALT in lean control mice and obese mice. Serum was recovered from lean (normal) mice or obese mice subjected to high fat diet for 8 weeks. Obese mice were treated with 25 μg OVA or omega-1 on days 0, 2 and 4. Serum was recovered on Day 6 and serum levels o AST and ALT analsyde by ELISA>f Data are representative of n=4-6 (+/−SEM) from 2 independent experiments. P<0.05 significant differences between obese mice treated with OVA or omega-1.

### EXAMPLES

### Example 1

### Method

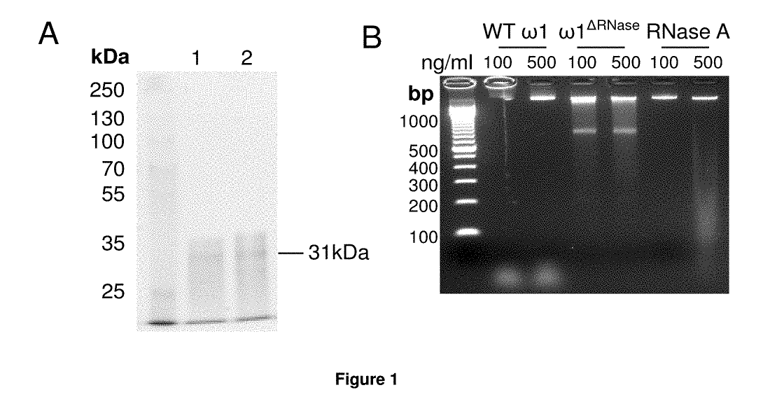

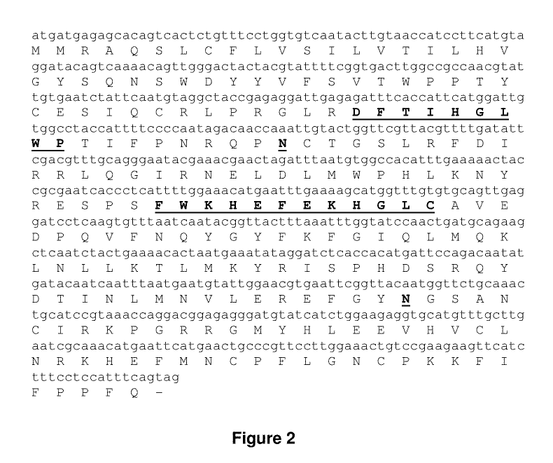

Recombinant omega-1 was expressed with a 6×His-tag in HEK293 cells that were transfected with the expression vector pSecTag2-omega-1. In addition a recombinant Omega-1 RNase mutant protein (ω1Δ^{RNase}), was prepared by mutating the Histidine 58 in CAS1 (FIG. 2) to Phenylalanine. Recombinant proteins were purified from culture supernatants by nickel-affinity and gel-filtration chromatography. Purified protein was subjected to detergent extraction, with recombinant omega-1 preparations having <0.5 EU per mg protein. The resultant ˜31 kDa protein was checked for purity by SDS-PAGE and western blotting using an anti-His tagged mAb (FIG. 1).

For all studies diet-induced obesity was initiated and maintained in 7-9 week old C57BU6J strain mice by feeding a 60% fat diet ad libitum for <8 weeks, during which time mice gain approximately 20% additional body weight (termed HFD), as described [10]. As a control for diet-induced obesity, mice were fed a nutritionally balanced diet containing 20% fat ad libitum which maintains a normal body weight gain with age (termed CD).

For acute treatment HFD and CD mice were treated with endotoxin-free recombinant omega-1 (25 μg in PBS i.p.) on days 0, 2 and 4; as a glycoprotein control HFD and CD mice were treated with endotoxin-free ovalbumin (OVA) (25 μg in PBS i.p.) on days 0, 2 and 4. Weight and condition were monitored daily. Metabolic parameters and cellular accumulation in the VAT were assessed on day 6, 2 days after the final treatment with recombinant omega-1, or on day 21, 16 days after the final omega-1 treatment. Chronic treatment involved administration of endotoxin-free recombinant omega-1 (25 μg in PBS i.p.) on days 0, 4, 8, 12, 16 and 20 with metabolic studies conducted on days 21.

Blood glucose was measured using a glucometer in mice fasted for 16 hours. Glucose tolerance was determined after i.p. injection of 2 g/kg glucose and blood glucose measured 30, 60 and 120 minutes to determine clearance from the blood. Serum triglyceride and liver enzyme (ALT; alanine transaminase, AST; glutamic oxaloacetate transaminase) levels were determined using commercially available kits from Abnova and Abcam respectively. Histological analysis of formalin fixed adipose tissue stained with hematoxylin and eosin allowed calculation of adipocyte area. Oil red O staining was performed on cryopreserved liver sections to determine lipid deposition in the liver.

To determine the cellular composition of the VAT, flow cytometric analysis was performed on a single cell suspension generated from VAT, with data collection on a CyAn ADP cytometer and data analysed using FlowJo software. To identify ILC2 cells were stained with BD Biosciences mAbs; CD8-APC (Ly-2), B220-APC (RA3-6B2), F4/80-APC (BM8), ICOS-PE (7E.17G9), Siglec-F-APC (E50-2440); eBiosciences mAbs; CD4-APC (RM4-5), CD11b-APC (M1/70), Gr-1-APC (RB6-8CS), FcER1-APC (MAR-1) and T1/ST2-FITC mAb (DJ8: MD biosciences). To identify eosinophils and AAM cells were stained with BD Biosciences mAbs; Siglec-F-PE (E50-2440), F4/80-APC (BM8), eBiosciences mAb; CD11b-PerCP (M1/70) and BioLegend mAb; CD206-PECy7 (C068C2).

Murine adipocytes were isolated from VAT after mechanical shredding and incubation with 1 mg/ml Collagenase D for 1 hour at 37° C. Adipocytes were collected from the surface of the media, washed in PBS supplemented with 2% FCS and resuspended at a density of 2×10⁶ cells/ml in RPMI supplemented with 2 mM L-glutamine, 100 U/ml penicillin and 100 μg/ml streptomycin. Human adipocytes were isolated from omental adipose tissue biopsies from patients undergoing elective abdominal surgery. Omental samples were processed to isolate adipocytes as described for mouse VAT samples. Mouse and human adipocytes were incubated with 500 ng/ml endotoxin-free recombinant omega-1 for 3 and 24 hours.

ELISA techniques were used to determine IL-4, IL-5, IL-13 and IL-33 levels in the peritoneal cavity in response to recombinant omega 1, 3 and 6 hours after treatment. IL-33 release from adipocytes in response to omega 1 was also determined in the culture supernatant by ELISA. All ELISAs were performed using duoset kits from R&D Systems, following the manufacturer's protocols.

### Results

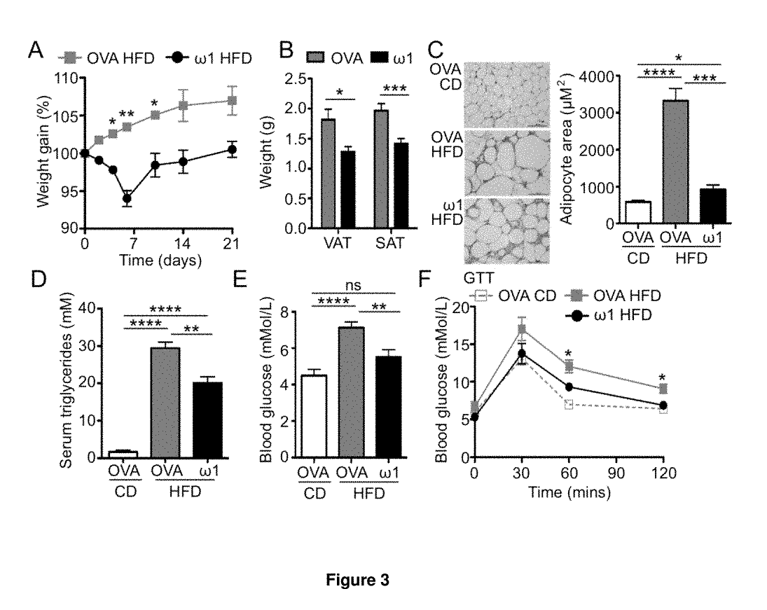

Obese mice, ˜30 g after being maintained on a HFD for >8 weeks, were treated with recombinant ω1 (25 μg per mouse i.p. injections on days 0, 2 and 4) and had a significant (P<0.01-0.05) transient weight loss relative to obese mice injected with control protein (OVA) (FIG. 3A). The weight loss in obese mice treated with ω1 was specifically associated with decreased adiposity as determined by a significant decrease in both visceral and subcutaneous white adipose tissue weight (FIG. 3B). In addition, the size of adipocytes was reduced size in ω1-treated mice (FIG. 3C). Treatment with ω1 also significantly (P<0.05) decreased serum levels of free triglyceride in obese animals (FIG. 3D).

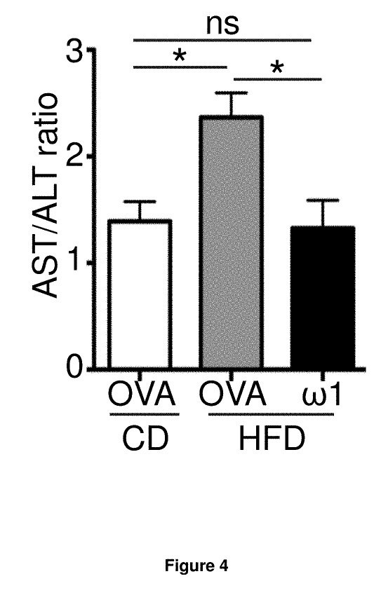

Importantly, treatment with ω1 did not induce anorexia or pyrexia in the mice (data not shown). In addition to weight loss and decreased adiposity, treatment with ω-1 significantly reduced fasting blood glucose levels in obese mice, to a point where blood glucose is no longer significantly elevated above levels seen in mice maintained on a 20% fat control diet (CD) (FIG. 3E). Furthermore, ω1-treated obese mice show significantly (P<0.05) improved glucose tolerance when compared to control OVA-treated mice (FIG. 3F). Obese mice develop liver steatosis, as indicated by elevated serum levels of ratios of hepatic enzymes glutamic oxaloacetate transaminase (AST) to alanine transaminase (ALT) in HFD fed mice relative to control diet fed mice (FIG. 4). Mice treated with ω1 [25 μg (1 mg/kg); 3 treatments day 0, 2, 4] had reductions in AST/ALT ratio (FIG. 4), indicating that following this short-term regimen ω1 improved the hepatic steatosis typically associated with obesity.

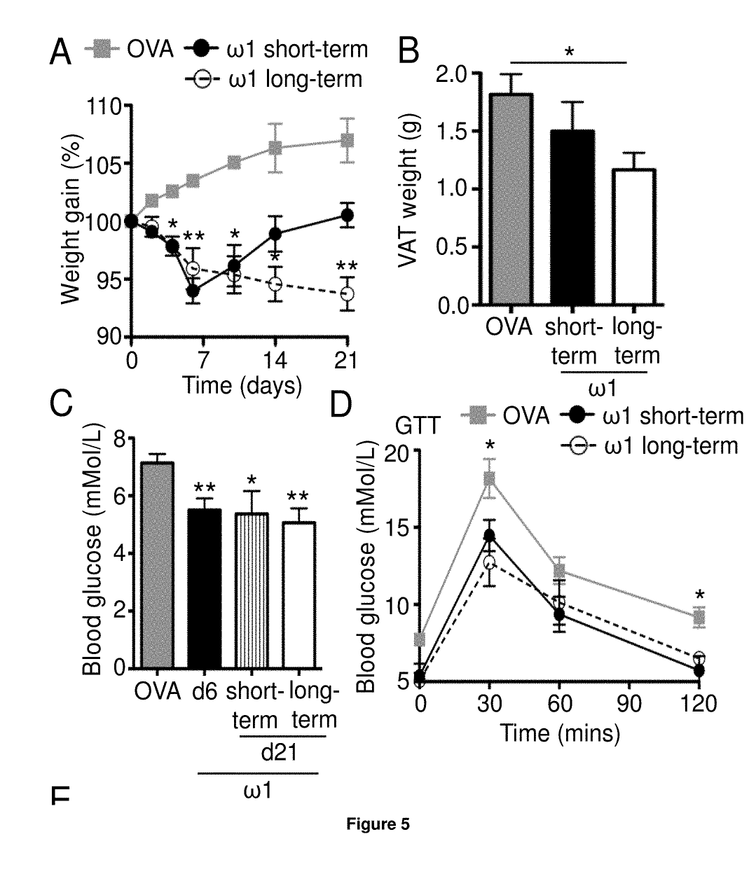

To assess the effects of ω1 treatment over time a long-term treatment regimen was employed. In mice treated with ω1 every 4 days for 20 days there was rapid weight loss, which was sustained throughout the treatment regimen (FIG. 5A), with decreased adiposity 21 days after commencement of treatment (FIG. 5B). Furthermore, long-term treatment sustained the decrease in basal blood glucose and improvement in glucose tolerance (FIG. 5C, D).

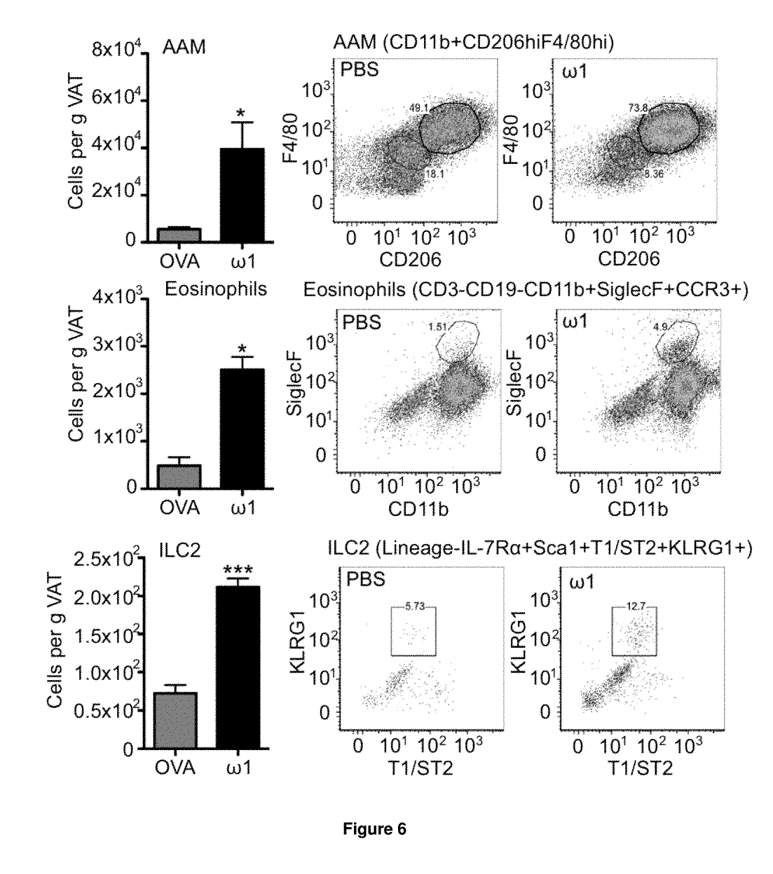

Studies have identified type 2 cells such as eosinophils, ILC2 and AAM as pivotal in promoting insulin sensitivity and improved glucose tolerance [3, 10, 11]. Treatment of obese mice with recombinant ω1 significantly increases accumulation of ‘anti-inflammatory’ type 2 cells in the adipose tissue of obese mice (FIG. 6).

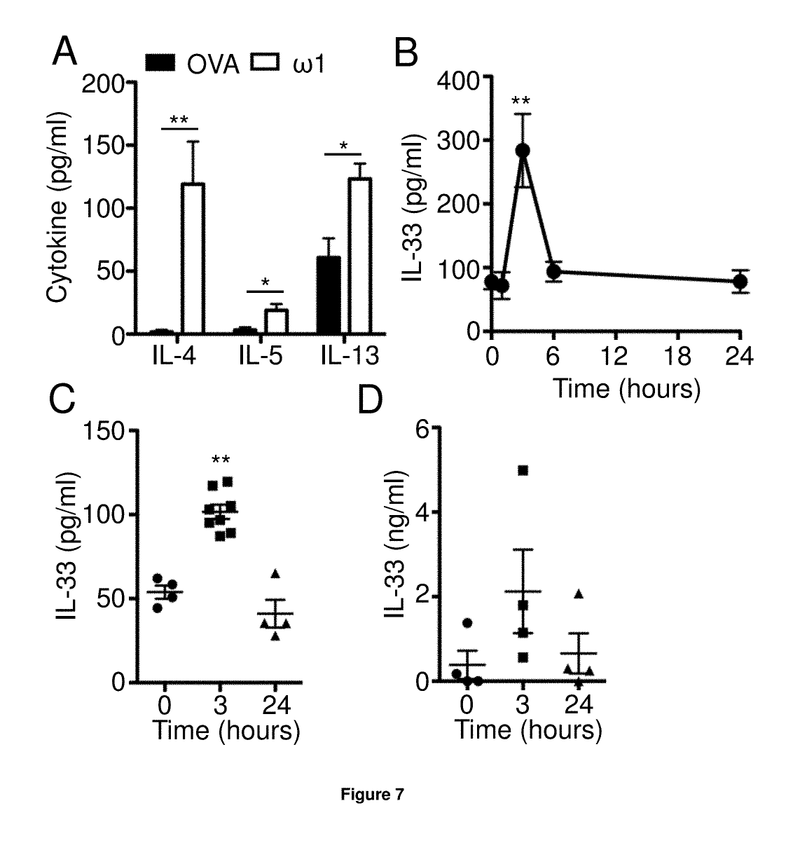

The ability of w 1 to induce type 2 cells, including eosinophils, ILC2 and AAM is due to the localized induction of type 2 cytokines including IL-4, IL-5, IL-13 and IL-33 (FIG. 7A, B). Obese mice were treated with ω1 i.p. and peritoneal lavage fluid collected at 1, 3, 6 and 24 hours. Levels of IL-4, IL-5, IL-13 and IL-33 were all induced in response to ω1. Furthermore, we identify ω1 as a potent inducer of IL-33 from both mouse (FIG. 7C) and human adipocytes (FIG. 7D).

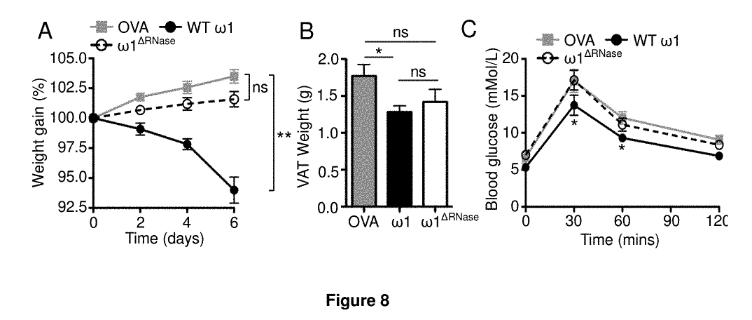

S. mansoni ω1 has been identified as a T2 RNase, a property shown to be integral to the ability of ω1 to induce a type 2 response [9]. Treating obese mice with ω1Δ^{RNase} did not induce significant weight loss, or a significant reduction in adiposity (FIG. 8A, B). Furthermore, ω1Δ^{RNase} did not improve glucose tolerance in obese mice (FIG. 8C). In contrast to the ω1Δ^{RNase} protein the intact recombinant ω1 (FIG. 1B) was efficacious in modulating these parameters in obese mice (FIG. 8A-C).

It will be understood that the invention is not limited to the embodiment hereinbefore described, but may be varied in both construction and detail within the scope of the claims.

### Example 2

### Method

It is in public domain that S. mansoni ω1 has been identified as a T2 RNase, a property shown to be integral to the ability of ω1 to induce IL-4 and IL-5 release.

A recombinant ω1 RNase-null (ω1Δ^{RNase}) mutant was generated, by substituting a phenylalanine residue in the RNase catalytic domain with a histidine residue (H58F) that was devoid of RNase activity.

### Results

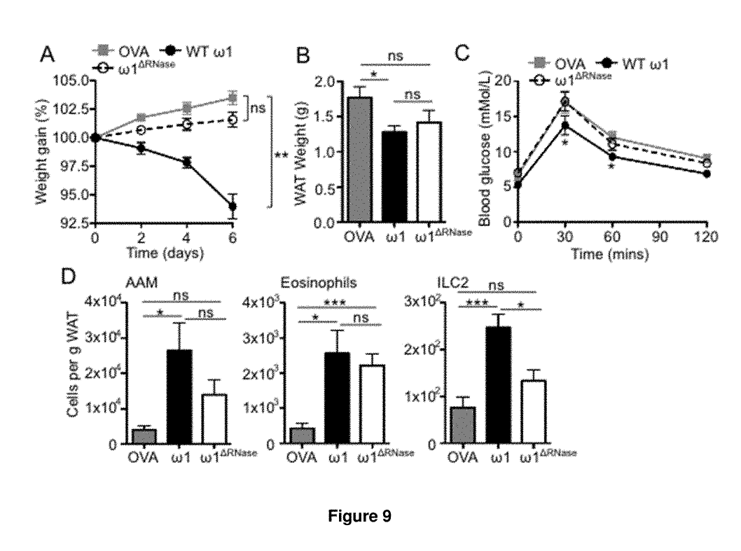

Treating obese mice with ω1Δ^{RNase} did not induce significant weight loss, or a significant reduction in adiposity (FIG. 9A,B). Furthermore, ω1Δ^{RNase} did not improve glucose tolerance in obese mice (FIG. 9C). The absence of functional RNase activity also diminished type 2 cell infiltration into the adipose tissue, with fewer ILC2 and AAM observed, although interestingly, eosinophil infiltration is still significantly (P<0.001) increased (FIG. 9D).

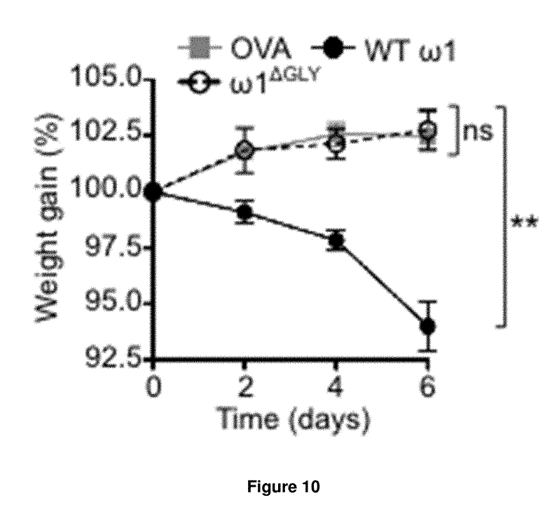

The function of ω1 is also known to be partly mediated through its binding to the surface of DCs via the mannose receptor (CD206). Using a recombinant ω1 with mutations in the sites responsible for glycosylation (N71/176Q; ω1Δ^{GLY}) and thus unable to bind to CD206, we show no effect on weight gain in the absence of the ability to bind to CD206 (FIG. 10).

### Conclusion

We have now confirmed that the weight loss induced by omega-1 is mediated by the known RNAse activity and glycosylation pattern of the molecule.

### Example 3 Hepatic Steatosis Assessment

### Method

The levels of the enzymes Alanine Transaminase (ALT) and Asparate Transaminase (AST) were quantified in the serum recovered from omega-1 or control (Ovalbumin; OVA) protein treated obese mice and lean mice to assess hepatic steatosis. The activity of both enzymes was quantified using kits from Abcam (Cambridge, UK), following the manufacturer's instructions.

### Results

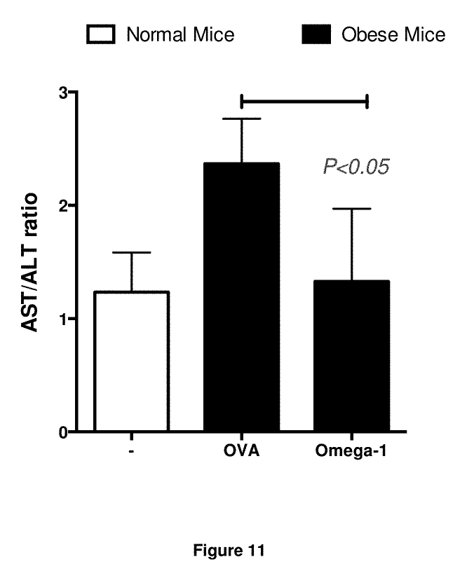

Results are displayed as a ratio of AST to ALT in lean control mice and obese mice and shown in FIG. 11.

### Discussion

Omega-1 has been reported to be hepatotoxic, with hepatocyte microvesicular damage developing when the native molecule is released from eggs that are deposited in the liver of mice infected with Schistosoma mansoni.

In contrast, we found that when recombinant omega-1 was injected into the peritoneal cavity of obese mice in addition to inducing weight loss and improving glucose tolerance it also reduced the ratio (AST:ALT) of the enzyme markers of hepatic steatosis. Thus, intraperitoneal injection of recombinant omega-1 does not cause hepatoxicity.

### Conclusion

One of the diseases that arise as part of the metabolic syndrome in man is non-alcoholic fatty liver disease with hepatic steatosis. In mice fed HFD-diet the obese state that develops is associated with hepatic steatosis, with hepatocyte microvesicular damage reflected by an elevated ratio of aspartate transaminase (AST) to alanine transaminase (ALT) enzymes in the serum. We found that obese mice treated with omega-1 had reduced hepatic steatosis as demonstrated by significantly (P<0.05) reduced circulating AST:ALT levels, comparable to non-obese mice, 6 days after initial treatment.

### The Invention Will Now be Described by the Following Non-Limiting Statements:

1. A compound or protein with ribonuclease activity or a fragment thereof that induces IL-33 release to initiate a type 2 response, preferably in adipose cells and/or tissues, for use in the treatment of diseases associated with fat accumulation.
2. The compound, protein or fragment thereof for use according to statement 1 which is a ribonuclease protein or a ribonuclease-like protein.
3. The protein or fragment thereof for use according to statement2 which is a ribonuclease protein of the T2 family.
4. The protein or fragment thereof for use according to any of the preceding statements which is a ribonuclease protein of the T2 family or fragment thereof comprising at least one or more RNAase catalytic domains.
5. The protein or fragment thereof for use according to any of the preceding statements which is a ribonuclease protein of the T2 family or fragment thereof and comprises at least a first conserved amino acid sequence (CAS1) comprising amino acid residues FTIHGLWPT and/or a second conserved amino acid sequence (CAS2) comprising amino acid residues PSFWKHEFEKHGLCAV.
6. The protein or fragment thereof for use according any of the preceding statements wherein the protein is Omega-1 protein or a fragment thereof.
7. The protein or fragment thereof for use according to statement6 wherein the protein is an Omega-1 protein or an Omega-1 protein fragment, preferably comprising at least part of amino acid residues 1 to 224, more preferably comprising at least one or more RNAase catalytic domains.
8. The protein or fragment thereof for use according to statement6 or 7 wherein the Omega-1 protein fragment comprises at least a first conserved amino acid sequence (CAS1) comprising amino acid residues FTIHGLWPT and/or a second conserved amino acid sequence (CAS2) comprising amino acid residues PSFWKHEFEKHGLCAV.
9. The protein or fragment thereof for use according any of the preceding statements with modified N-glycolysation sites or lacking N-glycolysation sites.
10. The protein or fragment thereof for use according any of the preceding statements further comprising a glycoprotein carrier.
11. The compound, protein or fragment thereof for use according to any of the preceding statements in the treatment of obesity and obesity-related disorders and/or inducing weight loss by decreasing the number of adipose cells after administration.
12. The compound, protein or fragment thereof for use according to any of the preceding statements in the treatment of metabolic disorders by restoring glucose and insulin homeostasis after administration.
13. The compound, protein or fragment thereof for use according to any of the preceding statements in the treatment of a liver disorder by decreasing the number of adipose cells in the liver after administration.
14. A pharmaceutical composition comprising the compound, protein or fragment thereof according to any of the preceding statements.
15. The compound, protein or fragment thereof or pharmaceutical composition according to any of the preceding statements for use as an adjuvant therapy.

### REFERENCES

1. Winer, S., et al., Normalization of obesity-associated insulin resistance through immunotherapy. Nat Med, 2009. 15(8): p. 921-9.
2. Exley, M. A., et al., Interplay between the immune system and adipose tissue in obesity. J Endocrinol, 2014. 223(2): p. R41-R48.
3. Wu, D., et al., Eosinophils sustain adipose alternatively activated macrophages associated with glucose homeostasis. Science, 2011. 332(6026): p. 243-7.
4. Pearce, E. J. and A. S. MacDonald, The immunobiology of schistosomiasis. Nat Rev Immunol, 2002. 2(7): p. 499-511.
5. Everts, B., et al., Omega-1, a glycoprotein secreted by Schistosoma mansoni eggs, drives Th2 responses. J Exp Med, 2009. 206(8): p. 1673-80.
6. Steinfelder, S., et al., The major component in schistosome eggs responsible for conditioning dendritic cells for Th2 polarization is a T2 ribonuclease (omega-1). J Exp Med, 2009. 206(8): p. 1681-90.
7. Dunne, D. W., F. M. Jones, and M. J. Doenhoff, The purification, characterization, serological activity and hepatotoxic properties of two cationic glycoproteins (alpha 1 and omega 1) from Schistosoma mansoni eggs. Parasitology, 1991. 103 Pt 2: p. 225-36.
8. Fitzsimmons, C. M., et al., Molecular characterization of omega-1: a hepatotoxic ribonuclease from Schistosoma mansoni eggs. Mol Biochem Parasitol, 2005. 144(1): p. 123-7.
9. Everts, B., et al., Schistosome-derived omega-1 drives Th2 polarization by suppressing protein synthesis following internalization by the mannose receptor. J Exp Med, 2012. 209(10): p. 1753-67, S1.
10. Hams, E., et al., Cutting edge: IL-25 elicits innate lymphoid type 2 and type II NKT cells that regulate obesity in mice. J Immunol, 2013. 191(11): p. 5349-53.
11. Molofsky, A. B., et al., Innate lymphoid type 2 cells sustain visceral adipose tissue eosinophils and alternatively activated macrophages. J Exp Med, 2013. 210(3): p. 535-49.

1-19. (canceled)

20. A method for the treatment of diseases associated with fat accumulation, comprising the administration of a ribonuclease protein of the T2 family, or a fragment thereof that induces IL-33 release to initiate a type 2, optionally in adipose cells and/or tissues, to a subject in need thereof.

21. The method according to claim 20 wherein the ribonuclease protein is Omega-1 protein or a fragment thereof.

22. The method according to claim 20 wherein the diseases associated with fat accumulation include obesity, obesity-related disorders and metabolic disorders.

23. The method according to claim 20 wherein the ribonuclease protein or a fragment thereof induces IL-33 release to initiate a type 2 response in adipose cells and/or tissues,

24. The method according to claim 20 wherein the ribonuclease protein or a fragment thereof comprises at least one or more RNAase catalytic domains.

25. The method according to claim 20 wherein the ribonuclease protein or a fragment thereof comprises at least a first conserved amino acid sequence (CAS1) comprising amino acid residues FTIHGLWPT and/or a second conserved amino acid sequence (CAS2) comprising amino acid residues PSFWKHEFEKHGLCAV.

26. The method according to claim 20 wherein the ribonuclease protein or a fragment thereof is Omega-1 protein or a fragment thereof.

27. The method according to claim 26 wherein the Omega-1 protein fragment comprises at least part of amino acid residues 1 to 224.

28. The method according to claim 26 wherein the Omega-1 protein fragment comprises at least one or more RNAase catalytic domains.

29. The method according to claim 26 wherein the Omega-1 protein fragment comprises
at least a first conserved amino acid sequence (CAS1) comprising amino acid residues FTIHGLWPT; and/or
a second conserved amino acid sequence (CAS2) comprising amino acid residues PSFWKHEFEKHGLCAV.

30. The method according to claim 20 wherein the ribonuclease protein or fragment thereof further comprises a glycoprotein carrier.

31. The method according to claim 20 for the treatment of obesity and obesity-related disorders and/or inducing weight loss by decreasing the number of adipose cells after administration.

32. The method according to claim 20 wherein the obesity related disorders are selected from heart disease, stroke, high blood pressure/hypertension, glucose disorders including diabetes (type 1 and type 2 diabetes mellitus), cancer, gallbladder disease and gallstones, osteoarthritis, gout, breathing problems, such as sleep apnea and asthma.

33. The method according to claim 22 for the treatment of metabolic disorders by restoring glucose and insulin homeostasis after administration.

34. The method according to claim 22 wherein the metabolic disorders associated with fat accumulation include type 1 and type 2 diabetes mellitus, high blood pressure/hypertension, nonalcoholic fatty liver disease, atherosclerosis, cancers, breathing problems including sleep apnea and cardiovascular diseases.

35. The method according to claim 22 wherein the metabolic disorders associated with fat accumulation is nonalcoholic fatty liver disease.

36. The method according to claim 20 for the treatment of a liver disorder by decreasing the number of adipose cells in the liver after administration.

37. The method according to claim 20 wherein the fat accumulation disorder is fatty liver disease.

38. A pharmaceutical composition comprising a ribonuclease protein of the T2 family, or a fragment thereof that induces IL-33 release to initiate a type 2, preferably in adipose cells and/or tissues.

39. The pharmaceutical composition according to claim 38 wherein the ribonuclease protein is Omega-1 protein or a fragment thereof.


SEQUENCE LISTING (4 sequences)

SEQ ID NO: 1 - DNA, 847 bp, Schistosoma mansoni
  CDS (16)..(690)
tgatttggattgatgatgagagcacagtcactctgtttcctggtgtcaatacttgtaaccatccttcatgtaggatacagtcaaaacagttgggactactacgtattttcggtgacttggccgccaacgtattgtgaatctattcaatgtaggctaccgagaggattgagagatttcaccattcatggattgtggcctaccattttccccaatagacaaccaaattgtactggttcgttacgttttgatattcgacgtttgcagggaatacgaaacgaactagatttaatgtggccacatttgaaaaactaccgcgaatcaccctcattttggaaacatgaatttgaaaagcatggtttgtgtgcagttgaggatcctcaagtgtttaatcaatacggttactttaaatttggtatccaactgatgcagaagctcaatctactgaaaacactaatgaaatataggatctcaccacatgattccagacaatatgatacaatcaatttaatgaatgtattggaacgtgaattcggttacaatggttctgcaaactgcatccgtaaaccaggacggagagggatgtatcatctggaagaggtgcatgtttgcttgaatcgcaaacatgaattcatgaactgcccgttccttggaaactgtccgaagaagttcatctttcctccatttcagtagttgcatgaagttgtttaatgaacgcactgattgactctgacaataacaaaaccgttcatgattattcatgtcacgtctgctctaacaaacagaaatcctcgtcatgaagttcatttgttcttaactaataaatatacattcgatcaaccaaaaaaaa

SEQ ID NO: 2 - protein, 224 aa, Schistosoma mansoni
MRAQSLCFLVSILVTILHVGYSQNSWDYYVFSVTWPPTYCESIQCRLPRGLRDFTIHGLWPTIFPNRQPNCTGSLRFDIRRLQGIRNELDLMWPHLKNYRESPSFWKHEFEKHGLCAVEDPQVFNQYGYFKFGIQLMQKLNLLKTLMKYRISPHDSRQYDTINLMNVLEREFGYNGSANCIRKPGRRGMYHLEEVHVCLNRKHEFMNCPFLGNCPKKFIFPPFQ

SEQ ID NO: 3 - protein, 9 aa, Schistosoma mansoni
FTIHGLWPT

SEQ ID NO: 4 - protein, 16 aa, Schistosoma mansoni
PSFWKHEFEKHGLCAV